(19) United States
(12) Patent Application Publication
(10) Pub. No.: US 2014/0371577 A1
(43) Pub. Date: Dec. 18, 2014

(54) ROBOTIC MEDICAL DEVICE FOR MONITORING THE RESPIRATION OF A PATIENT AND CORRECTING THE TRAJECTORY OF A ROBOTIC ARM

(71) Applicant: MEDTECH, Castelnau le Lez (FR)

(72) Inventors: Pierre Maillet, Saint Aunes (FR); Bertin Nahum, Baillargues (FR); Fernand Badano, Villeurbanne (FR); Patrick Dehour, Crespian (FR)

(73) Assignee: MEDTECH, Castelnau le Lez (FR)

(21) Appl. No.: 14/365,284

(22) PCT Filed: Oct. 31, 2012
(86) PCT No.: PCT/FR12/52532

§ 371 Date: Jun. 13, 2014

(30) Foreign Application Priority Data
Dec. 30, 2011 (FR) 11 62555
Apr. 27, 2012 (FR) 12 53919

(51) Int. Cl. A61B 19/00; A61B 17/00; A61B 5/06; A61M 16/00

(52) U.S. Cl. CPC A61B 19/2203; A61B 19/5244; A61M 16/0057; A61B 19/5212; A61B 17/00234; A61B 5/066; A61B 2017/00699; A61B 2019/5238; A61B 2019/5276; A61B 2019/5257
USPC 600/424

(57) ABSTRACT

The robotic medical device for monitoring the respiration of a patient and correcting robotic trajectory includes at least one robotic arm; a mechanical ventilator, to which the respiration of a patient is subjected; and a recorder for, on the basis of time, times during mechanical ventilation when the patient is in an original position and a high position. There is also a device for capturing images of an anatomical region of the patient, the triggering of the device being synchronized with the times recorded in the original position and the high position. There is also a device for calculating a three-dimensional displacement vector of the region between the original and high positions and a device for correcting the trajectory of the robotic arm on the basis of a calculated three-dimensional vector.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### BACKGROUND OF THE INVENTION

1. Field of the Invention

The present invention falls within the medical field, in particular in the surgical methodology during the preparation and conduction of surgery operations.

The invention specifically relates to anatomical medical imaging, in order to carry out robotic-assisted surgery operations.

The invention relates to a robotic medical device for monitoring the respiration of a patient and correcting the robotic trajectory.

The present invention will find a preferred, but in no way limited, application to surgery operations in the anatomic area of the rachis.

It should be noted that the invention will be described according to a particular example of operation at the level of the lumbar rachis, at the level of the anterior curvature of the lordosis of the spine. However, the invention can be used for an operation at the level of the upper and lower cervical rachis, of the lower back or thoracic rachis, as well as the sacral rachis and the coccyx.

In this context, a major problem during a robotic-assisted operation lies in the management of anatomical movements of the patient due to his own breathing. In particular, the breathing depends on the activity of the diaphragm generating chest and lung movements contributing to the gas exchanges. This muscular activity causes a deformation of the collateral anatomical parts, such as the abdomen, but especially the rachis. The magnitude of this distortion depends on the minute ventilation (MV), depending on its volume and its frequency, but also on the position of the patient, i.e. standing, sitting, but also lying on his stomach, back or a side.

In the case of an operation on the rachis, the latter moves to a larger extent for the thoracic vertebrae and to a lesser extent for the lumbar vertebrae.

In order to limit these movements during a lumbar surgery, for example, when the access path permits such, the patient is lying on his stomach, taking care to leave the movements of the belly free below the level of the chest region. The patient is then immobilized in this position by mechanisms and accessories of the operating table. This particular prone position permits to significantly reduce the magnitude of the movements of the lumbar rachis.

Careful operations are thus performed in the area of the lumbar rachis such as laminectomy (narrow channel), radicular release (herniated disc), arthrodesis (combining vertebrae by screwing into a pedicle), kyphoplasty and vertebroplasty injecting cement into the vertebral body.

However, breathing generates periodic movements of the lumbar rachis of a few millimeters, which the surgeon is then forced to compensate for thanks to his dexterity and his visual acuity

This obligation to compensate is even more important with the use of a robotic system that replaces the hand of a neurosurgeon. Indeed, robotics should ensure improved accuracy of the gestures in order to make them secure (such as accurate drilling of the pedicle, identification of the anatomy through minimally invasive surgery or percutaneous endoscopy, or the definition of secure areas, in order to avoid damaging the spinal cord, veins, nerves). On the other hand, the robot must accompany the predictable movements of the anatomy by anticipating them, like the surgeon's hand and eye, this with a speed adapted to the speed of the target, for otherwise damage may be more important with the robot following its program in a static environment.

2. Description of Related Art Including Information Disclosed Under 37 CFR 1.97 and 37 CFR 1.98

Presently, a simplifying approach consists in using a laser or ultrasonic rangefinder, the beam of which is oriented, in its vertical direction, onto the skin covering a vertebra of the backbone. The amplitude of the displacement of said vertebra is thus recorded, but along a single vertical axis. In addition, with a measurement of a surface on the skin, it is not possible to accurately determine whether the vertebra moves like the skin, neither in which directions in space.

Another existing solution consists in screwing a marker into the patient's backbone, to which the optics of a three-dimensional measuring system is oriented. This system permits to capture in real time the coordinates in space of said marker. These coordinates thus permit to monitor the movement of the vertebra, on which the marker is positioned. Then, an algorithm calculates on a computer the compensations in the reference mark within which said robotic arm moves, in correlation with the three-dimensional medical imaging performed prior to the operation.

However, the measured displacement is limited in accuracy to the vertebra, on which the reference mark is placed, whereby differences can be observed in the movements of the vertebrae relative to each other, as well as of the collateral organs. It would then be necessary to position a marker on each vertebra or at the level of the vertebrae around the anatomical area of the operation. Let's recall in this respect that this solution has the drawback of being invasive and that multiplying the number of markers is therefore not a satisfactory solution in the context of movements of several vertebrae.

By way of an example, a known solution is described in US 2010/063514. This is a device and a method for monitoring the respiratory movements of a patient, in the context of invasive medical operations at the level of an anatomical area of the body of said patient.

To this end, image acquisitions are performed continuously, through X-rays or ultrasounds. Then the recorded images permit to calculate the movements of the anatomical area involved, and to obtain a curve of said movements, preferably a periodic curve.

In addition, the monitoring may use a respirator. In this case, the internal clock of said respirator can serve as a triggering signal for the pre- or per-operative imaging. It simply consists in using the internal clock of the respirator as a trigger. Therefore, the movements thus detected can permit the correction of the trajectory of a surgical tool in synchronism with the breathing of the patient, in particular the trajectory of a robot.

However, this document specifies in no way the technique used, in particular the calculation performed, which permits, based on the taking of successive images and continuously, to obtain the desired trajectory correction.

### SUMMARY OF THE INVENTION

The aim of the present invention is to cope with the drawbacks of the state of the art by providing a device permitting to simulate the movements of the lumbar rachis under the action of the respiration, in order to correct the movements of a robotic system, namely a robotic arm, supporting surgical tools and active processing means (laser-like means or radiating means for therapeutic purposes).

The invention pretends to be able to measure these movements for the robotic arm to automatically adapt to them and even to be able to anticipate them, in order to maintain the improvement of the robotic accuracy relative to that of the surgeon, while accompanying said movements with a speed of execution corresponding to the speed of the target.

In addition, the invention provides a solution having the advantage of being non-invasive.

To this end, the invention relates to a robotic medical device for monitoring the respiration of a patient and correcting the trajectory of a robotic arm, comprising:

at least one robotic arm;

a mechanical ventilator, which the respiration of a patient is subjected to;

means for recording depending on the duration of the time instants during said mechanical ventilation where said patient is in an original position corresponding to the position of the patient at the end of expiration of the gases until the next insufflation of gases and in a high position corresponding to the maximum position at the end of the insufflation;

means for digitally capturing images of an anatomical area of said patient, the triggering of said means for capturing being synchronized with the instants recorded in said original position and in said high position;

means for calculating at least one three-dimensional displacement vector of said area between said original and high positions;

means for correcting the trajectory of said robotic arm depending on each calculated three-dimensional vector.

Such a solution relies in the first place on that the operations on the rachis are generally performed under so-called “general” anesthesia. Therefore, the ventilation of the patient is performed by means of a respirator ensuring a mechanical ventilation. Under these circumstances, it is then possible to know accurately and repetitively the breathing parameters of the anaesthetized patient.

The means implemented in the invention take these parameters into account and interpret them in order to compensate for the movement of the robotic arm depending on the patient's respiratory movements.

In particular, the invention provides as a matter of fact for recording two different positions at two different time instants. These positions are selected among all possible positions during the movement of the patient's body. These are in fact the extreme positions of displacement of the anatomical area, which coincide with the insufflation and expiration generated by the automatic artificial ventilator.

In addition, the invention integrates means permitting to detect the instants at which the patient is in these positions, using the operating parameters of said respirator. These very means then permit to control the triggering at these specific moments, without using the internal clock of said respirator as a trigger. In brief, the triggering of capture of images is performed synchronously thanks to the previously made recording. Thus, the invention implements a time synchronization different from the simple setting according to the internal clock of the respirator.

In addition, the recording occurs intermittently, by different images taken at different time instants, so as not to subject the patient unduly to waves and radiation.

Furthermore, the device according to the invention uses means for calculating a displacement vector between the two images taken at the two time instants. This vector is used to correct the trajectory of the robotic arm, through adapted means for transmitting this correction to said arm.

According to further features, such a device is characterized in that said means for capturing comprise ultrasonic sensors, the latter being positioned into contact with said anatomical area.

It should be noted that this measurement by ultrasounds can consist of ultrasound scanning.

Advantageously, said means for capturing comprise a fluoroscope.

In addition, said device comprises means for superimposing the images captured by said fluoroscope and said calculating means comprise, on the one hand, a module for determining at least one two-dimensional vector of displacement of said anatomical area from the superposed images and, on the other hand, a module for overlapping said two-dimensional vectors, in order to obtain said three-dimensional vector.

Preferably, said superimposing means include means for computer-processing said captured images by segmenting the contour of at least one anatomical element of said anatomical area appearing in each picture and said superimposition consists in superimposing said processed contours.

In particular, said computer-processing means comprise a manual pointing interface.

Moreover, said means for correcting the trajectory of the robotic arm comprise a computer module for resetting said captured anatomical images by matching them with a previously captured medical imaging, said resetting being performed with imaging operations preferably made in said original position.

It should be noted that it is possible to use alternative technical capturing or anatomic measuring means to obtain an equivalent result.

Further features and advantages of the invention will become clear from the following detailed description of the non-restrictive embodiments of the invention, with reference to the attached drawings.

### BRIEF DESCRIPTION OF THE DRAWINGS

FIGS. 1A and 1B are photographic illustrations representing two examples of fluoroscopic pictures corresponding to anatomical captures at the moment where the patient is in a given position, said two picture illustrations being mutually captured in the lateral plane and the anterior/posterior plane.

FIGS. 2A and 2B isolated photographic illustrations representing two examples of the processing step of reciprocally segmenting a first lateral picture illustration and a second anterior picture illustration.

FIG. 3 is a schematic view representing an example of superposition of the contours of two segmentations in the lateral plane between an original position and a high position.

### DETAILED DESCRIPTION OF THE DRAWINGS

The present invention relates to a robotic medical device for monitoring the respiration of a patient and correcting the trajectory of a robotic arm.

Under such a procedure, the patient is anesthetized. The curarisation affects the muscle function, among which the activity of the diaphragm, up to paralysis. Mechanical ventilation is then implemented, which ensures the ventilation of the patient. Therefore, the device comprises a mechanical ventilator, which the respiration of a patient is subjected to. Thus, the patient fully depends on the machine, which will always insufflate the same volume of air in a precise and indefinitely reproducible timing, subject to a stable pulmonary physiology.

In this context, the thoracic, abdominal, back movements, depending on the position of the patient and due to the mechanical ventilation, will always be reproducible and therefore predictable. For general anesthesia, mechanical ventilation is generally performed in Controlled Volume, we can also use Controlled Pressure.

In an operating mode of a ventilation in volume mode, the volume is a set value, which is characterized by a constant flow rate during a constant insufflation time.

Generally, the ventilation cycle is adjusted according to several parameters: the insufflation time or duration Ti adjusted to one third of the cycle, and the expiration time or duration Te adjusted to two thirds of the cycle. The expiration of gases by the patient corresponds at least to half the expiration time allocated and is invariable.

Thus, during the insufflation time Ti, the patient's lungs inflate, causing a deformation of the collateral anatomical parts, up to a limit that will be reached at the end of the inspiration time. Conversely, during expiration by the patient, his lungs deflate by themselves, due to their elastic properties, causing a deformation of the collateral anatomical parts, to finally return to their original position at the end of the expiration of the gases, in less than the allotted expiration time Te.

Between the end of the expiration of gases by the patient and until the next insufflation, the patient is theoretically immobile in a so-called “original” position.

By way of an example, for an adult a ventilation frequency of fifteen cycles per minute results into a total duration of four seconds per cycle for one second of insufflation and an allocated expiration time of three seconds. The expiration of the gases will be of about two seconds. Thus, the time of immobility will be of one second.

It should be noted that substantial adjustments of the respiration frequency F, in the form of a decrease or an increase of the ventilation cycle time, while maintaining the same insufflation time, can significantly increase the time of immobilization of the patient, by means of a satisfactory ventilation quality during a time determined and found acceptable by the anesthetist. One can even consider, in order to increase the time of immobilization, a decrease of the insufflation time Ti, while increasing the flow rate accordingly, in order to maintain the same current volume reference, at constant respiration frequency.

First of all, according to a first essential feature, during the operation of the device according to the invention, a monitoring of the patient's ventilation is performed in order to determine when the patient is immobile in its original position, when the deformation begins, when it reaches its maximum in a so-called “high” position and when the respiratory movement ends.

To this end, the mechanical lung ventilators generally use flow-rate and pressure sensors, an internal clock for monitoring said parameters, namely the gas insufflation time Ti, the gas expiration time, the allocated expiration time Te. Thus, sensors placed in the circuit of the mechanical ventilation system permit to perform the measuring of said parameters in real time and continuously. It is then possible, through an appropriate processing, to know when the patient is immobile, when the movement of the rib cage starts, when the movement reaches its maximum amplitude, when the rib cage returns to its original position, like all other collateral anatomical parts. This processing is performed by recording means, depending on the time, on the time instants during said mechanical ventilation at which said patient is in these precise and well-defined original and high positions.

Therefore, the invention records the time instant to at which the patient is in his original position and the time instant th when the patient is in his high position.

More specifically, said means record a periodic curve, which will permit to determine the resting or original position, then the high position of the patient over time, knowing that the parameters Ti, Te and F of the lung ventilator are perfectly known and invariable.

By way of an in no way restrictive example, during an operation, according to the usually observed practices, for a frequency of twelve cycles of 5 seconds each, a Ti of 1.7 seconds and a Te of 3.3 seconds are measured, with a period of immobility of 0.3 seconds.

Based on these temporal data, the invention advantageously provides for measuring in space the anatomical position when the patient is in both original and high positions, without trying to accurately measure neither the amplitude nor the deformation vector of the spine, or of a lumbar vertebrae in particular.

To this end, the device according to the invention comprises means for digitally capturing anatomical images of said patient, the triggering of said capturing means occurring depending on said periodic curve. In particular, the triggering of said capturing means is synchronized with the time instants recorded in said original position and in said high position. In other words, these captures occur at least at time instant to when the patient is in his original position and at least at time instant th when the patient is in his high position.

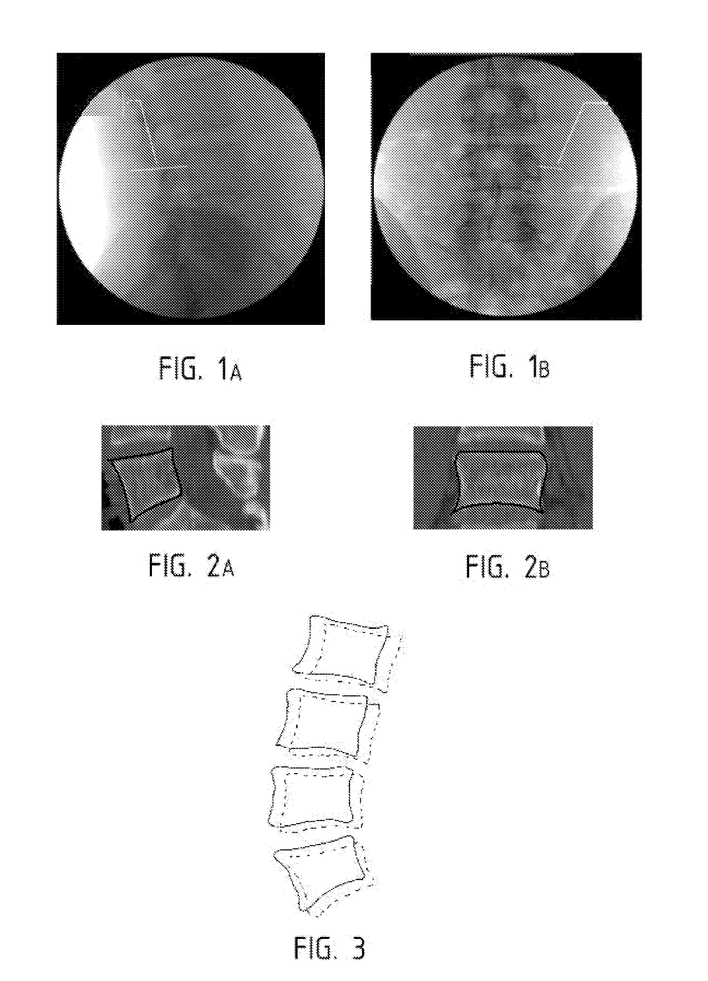

According to the preferred embodiment, these steps of capturing use a fluoroscopic technology. Thus, each capture is performed by taking at least one picture by means of a fluoroscope: for example a picture in the lateral plane (from the side or in profile), the most representative of the displacement of the rachis during breathing. A second picture in the anterior/posterior plane (from the front) permits to take the horizontal movements into consideration, as can be seen in both FIGS. 1A and 1B.

It should be noted that other capturing angles can be considered, where the angle of viewing of the plane of each picture can be changed depending on the position of the patient or the anatomical elements to be captured.

In brief, in the implementation of the device according to the invention, this step consists, once the patient has been anesthetized, immobilized and positioned on the operating table (in the prone position), then the recording of the periodic ventilation curve has previously been prepared, in triggering the fluoroscope for two first pictures, preferably lateral and anterior pictures, by synchronizing with the original position of the patient. Then, it will trigger two second pictures by synchronizing with the high position.

According to another alternative or complementary operating mode, these steps of capturing may be performed by ultrasounds. The device then comprises ultrasound sensors positioned into contact with the anatomical area of the patient, in front of the vertebrae involved by the action, with a controlled and adequate application force, thanks to a force sensor. The ultrasonic measurements permit to capture said positions of the vertebra.

It should be noted that the collection by ultrasounds of the measurements of the point of the vertebra by means of one or more ultrasonic sensors is performed during the immobility of the patient, namely in the original position at the end of the expiration of gases by the patient; then, in synchronization in the high position. In addition, based on the measurements in original and high positions, it is possible to accurately extrapolate the intermediate positions, without generating significant errors relative to the accuracy of recording and the robotic system. Such an extrapolation permits to eliminate a multiplication of measurements by ultrasound, which is however possible.

Based on the first and second pairs of so obtained fluoroscopy pictures, a computer processing is performed.

This processing consists in the first place in segmenting on each picture the two-dimensional contour of a dedicated anatomical element, in this example visible in FIGS. 2A and 2B, which show the vertebral body of the vertebra involved. It is also possible to segment several anatomical elements, in particular the contours of several vertebrae.

Based on the so obtained contours, it is possible, for each pair of pictures, preferably lateral and anterior pictures, to perform a superposition of the contours in both original and high positions. The following image shows a superposition of the contours of the two positions in the lateral plane, as can be seen in FIG. 3, which shows in solid line the original position and in dotted line the high position.

To this end, said device comprises means for superimposing the pictures captured by said fluoroscope and said calculating means comprise, on the one hand, a module for determining at least one two-dimensional displacement vector of said anatomical area based on the superimposed pictures and, on the other hand, a module for overlapping said two-dimensional vectors, in order to obtain said three-dimensional vector.

More specifically, said superimposing means comprise means for data processing said captured images, namely for segmenting the contour of at least one anatomical element of said anatomical area appearing in each image and said superposing means consist in superposing said so processed contours.

In addition, said data-processing means can comprise a manual pointing interface, permitting the practitioner to define specific points of the anatomical area, in particular points of the contour, e.g. of a vertebra, in order to guide the automatic segmentation carried out.

Based on these superimpositions, one per pair of preferably lateral and/or anterior pictures, the device permits to determine a two-dimensional displacement vector for each of the vertebrae in a respectively lateral (vertical or substantially vertical) and anterior (horizontal or substantially horizontal) plane.

Moreover, based on these lateral and anterior two-dimensional vectors, their overlapping permits to obtain a three-dimensional displacement vector for each vertebra. Thus, the device comprises means for calculating at least one three-dimensional displacement vector of said area between said original and high positions.

Then, based on these 3D vectors, the invention comprises a step of construction of a temporal and three-dimensional movement simulation of each vertebra, coordinated with the parameters F, Ti and Te given by the lung ventilator. Such a simulation can namely consist of at least one curve representing the movement of one or several points of each vertebra.

In addition, based on the contours in original and high positions, it is possible to accurately extrapolate the intermediate positions, without generating significant errors relative to the accuracy of recording and of the robotic system. Such extrapolation permits to eliminate a multiplication of the fluoroscopic pictures, which is however possible, but which subjects the patient and the staff to an increased and harmful exposure to X-rays.

It should be noted that the four original lateral, high lateral, original anterior and high anterior pictures chosen for the example are set in a identical known reference mark, that of the fluoroscope, identical to that of the robotic system. To this end, the target spotter of said fluoroscope can be carried directly by a robotic arm.

Therefore, it is possible, based on the three-dimensional movement simulation obtained, to correct and change, even by anticipation, the trajectory of intervention of the robotic system. Thus, the device comprises means for correcting the trajectory of said robotic arm depending on each calculated three-dimensional vector.

More specifically, said means for correcting the trajectory of the robotic arm comprise a computer module for resetting said anatomical images captured by matching with a previously captured medical imaging. Indeed, the existing robotic systems use a matching or registration of three-dimensional medical imaging, namely proceeding from a scanner or MRI, for “Magnetic Resonance Imaging”, and two-dimensional imaging, as fluoroscopy.

First of all, the 3D imaging performed pre-operatively permits to perform a planning of the operation, in particular of the operative procedures and of the paths that will be followed by each robotic arm. On the other hand, the per-operative 2D imaging permits to take control pictures, in order to ensure the positioning of the patient's anatomy, as mentioned above, and pass this position to the surgical instruments, but also to know their positions in a 3D reference mark of the browser or robotic system. In brief, the registration of the 3D and 2D images is a way to cause the tool to travel in the pre-operative 3D imaging, and vice-versa.

In this context, precautions are taken during a scanning, in order to make imaging in which the patient's movements or breathing will not degrade the result. The patient is then requested not to move, and some systems are capable of correcting the respiratory movements. The 3D imaging is thus considered as the result of an immobile patient with blocked breathing. This immobility is particularly true for the lower portion of the rachis, the lumbar area, when the patient is lying on his back. The pre-operative 3D imaging thus serves as a reference.

As mentioned above, when the patient is placed in surgical (prone) condition, the position of a vertebra will depend on the respiratory movement. In the case of the per-operative 2D fluoroscopy, this dependence continues logically. Thus, the per-operative control 2D fluoroscopic imaging system serves as a link for collecting the imaging and the robots in the same reference mark. It is also understood that the operation of resetting the robotic arm, through the target spotter, in the reference mark of the fluoroscopic image will occur during the immobility of the patient, i.e. in original position.

Therefore, when the pre-operative 3D imaging and the 2D fluoroscopy must be matched, the invention provides for using preferably the lateral and anterior pictures in the original position of the patient. This matching can be performed through known software, namely surface resetting software.

Based on this previous calibration of the 2D fluoroscopy images, through the identification and the matching of the original position during the time of total immobility of the patient, the invention then corrects in real time the reference mark of the original registration using said simulation of the vertebral movements, this during the stereotactic surgical operation, which can, in turn, also be performed by a robot.

This functionality permits to make sure the body area of interest is immobile, which is defined as the original position during the operation of registration. In addition, it permits to correct the resetting in real time due to the respiratory movements of the patient.

Thus, the robotic medical device for monitoring the respiration of a patient and correcting the robotic trajectory according to the present invention ensures, through a real-time analysis of the mechanical and automatic ventilation of a patient, the measuring of the original position of anatomical immobility and the measuring of a high position of maximum deformation, a simulation of the anatomical area of interest, particularly the vertebrae, which pretends to be reproducible during the successive respiratory cycles during a surgical operation. Such a simulation then permits to correct in space and time the reference mark of working of the robot on the anatomical area of interest, with a sub-millimeter spatial accuracy and a temporal accuracy within some thirty milliseconds.

It should be noted that the description of the present invention is applied by way of an example to the lumbar rachis, but may extend to any other adequate portion of the human anatomy of the rachis.

1. Robotic medical device for monitoring the respiration of a patient and correcting the robotic trajectory, comprising:
a robotic arm;
a mechanical ventilator, subject to respiration of said patient;
means for recording duration of time during mechanical ventilation from an original position of said mechanical ventilator corresponding to position of said patient at an end of expiration of gases to a raised position of said mechanical ventilator corresponding to a maximum high position of said patient at an end of insufflations of gases;
means for digitally capturing images of an anatomical area of said patient, wherein triggering said means for digitally capturing images is synchronized with time recorded in said original position and in said raised position;
means for calculating at least one three-dimensional displacement vector of area between said original position and said raised position; and
means for correcting trajectory of said robotic arm depending on each calculated three-dimensional vector.

2. Device according to claim 1, wherein said means for digitally capturing images comprise ultrasonic sensors, the sensors being positioned into contact with said anatomical area.

3. Device according to claim 1, wherein said means for digitally capturing images comprise a fluoroscope.

4. Device according to claim 3, further comprising:
means for superimposing pictures captured by said fluoroscope, wherein said means for calculating at least one three-dimensional displacement vector comprise a module for determining at least one two-dimensional displacement vector of said anatomical area based on superposed pictures and a module for overlapping said two-dimensional vectors, in order to obtain said three-dimensional vector.

5. Device according to claim 4, wherein said means for superimposing pictures comprise means for data-processing images captured by segmentation of a contour of at least one anatomical element of said anatomical area appearing in each image, each contour being superimposed.

6. Device according to claim 5, wherein said means data-processing images comprise a manual pointing interface.

7. Device according to claim 1, wherein said means for correcting trajectory of said robotic arm comprise a computer module for resetting anatomical images captured by matching with a previously captured medical imaging, wherein resetting is performed with imaging operations performed in said original position.
